(19) United States
(12) Patent Application Publication
(10) Pub. No.: US 2019/0076464 A1
(43) Pub. Date: Mar. 14, 2019

(54) CYTIDINE DEAMINASE INHIBITORS FOR THE TREATMENT OF PANCREATIC CANCER

(71) Applicants: INSERM (OMSTOTIT NATIONAL DE LA SANTÉ ET DE LA RECHERCHE MÉDICALE), Paris (FR); UNIVERSITÉ PAUL SABATIER TOULOUSE III, Toulouse (FR)

(72) Inventors: Pierre CORDELIER, Toulouse Cedex 1 (FR); Marion GAYRAL, Toulouse Cedex 1 (FR)

(21) Appl. No.: 16/084,830

(22) PCT Filed: Mar. 15, 2017
(86) PCT No.: PCT/EP2017/056160

§ 371 Date: Sep. 13, 2018

(30) Foreign Application Priority Data
Mar. 16, 2016 (IB) PCT/IB2016/001443

(51) Int. Cl. A61K 31/7115; A61P 35/00; A61K 31/713; C12N 15/62; A61K 31/7068; G01N 33/50; A61K 48/00

(52) U.S. Cl. CPC A61K 31/7115; A61P 35/00; A61K 31/713; A61K 48/005; A61K 31/7068; G01N 33/5011; C12N 15/62

(57) ABSTRACT

The present invention relates to methods and pharmaceutical compositions for use in the treatment of pancreatic cancer in a subject in need thereof. The inventors showed that targeting cytidine deaminase sensitizes cancer cells to chemotherapy (gemcitabine dFdC) both in vitro and in vivo in experimental models of PDA, with very high efficacy. To their surprise, CDA targeting in the absence of chemotherapy strongly alters cell proliferation and tumor progression. In particular, the present invention relates to a method for treating pancreatic cancer in a subject in need thereof comprising administering to the subject a therapeutically effective amount of a cytidine deaminase inhibitor in combination with an anti-pancreatic cancer treatment selected from the group consisting of CHK1 inhibitor, WEE1 inhibitor, ART inhibitor, DHODH inhibitor or gene therapy.

### FIELD OF THE INVENTION

The present invention relates to methods and pharmaceutical compositions for use in the treatment of pancreatic cancer in a subject in need thereof.

### BACKGROUND OF THE INVENTION

Pancreatic ductal carcinoma (PDA) is the most common type of pancreatic cancer¹. Despite decades of intense efforts from researchers and clinicians, PDA remains a challenge to treat, with 5 year rates survival lower than 6% for subjects with cancers of all stages¹. Most PDA is identified at a late stage, when surgical intervention is not possible. Even with complete resection and negative results from analyses of tumour margins, long-term survival after surgery is poor; tumors recur in virtually all subjects. To put this into perspective, PDA is estimated to become one of the top three leading cause of cancer-related death by 2030².

Progress in the treatment of PDA has been incremental. Combination cytotoxic therapies such as FOLFIRINOX³, along with gemcitabine⁴ and albumin-bound paclitaxel⁵, have provided meaningful gains, but there is lot of needs for improvement. The only targeted agents approved in the treatment of PDA is the EGFR inhibitor Erlotinib (Tarceva), which given in combination with gemcitabine, only slightly increases overall survival time compared with gemcitabine alone⁶. Taken together, the current treatment approaches for PDA increase survival times of subjects in weeks to months.

In this dismal context, the inventors have elected cancer gene therapy as a promising approach for PDA management⁷. The inventors conducted the first-in-human clinical trial, based on the use of non-viral vectors to transfer anticancer genes that sensitize PDA to gemcitabine⁸. This early phase clinical trial demonstrates that intratumoral gene delivery is safe and feasible in subjects with unresectable PDA. In addition, a population of subjects with locally advanced tumors benefited from this treatment, with two subjects surviving for up to two years following gene therapy⁸. A phase II clinical trial is under preparation.

While leading-edge, this trial also highlights the need to further characterize the molecular mechanisms involved in the resistance to treatment. Accordingly, the inventors have interrogated their clinical samples for the expression of key proteins involved in resistance of cancer cells to gemcitabine. The inventors found that cytidine deaminase (CDA) was the only gene (i) upregulated in resected PDA samples compared to normal parenchyma, (ii) overexpressed in microbiopsies from locally advanced and metastatic PDA resisting to therapy and (iii) detectable in microbiopsies of PDA subjects treated by gene therapy (FIG. 1).

Cytidine deaminase (CDA) is a key enzyme of the pyrimidine salvage pathway that catalyzes the hydrolytic deamination of cytidine and deoxycytidine to uridine and deoxyuridine, respectively⁹. In PDA, gemcitabine is inactivated primarily by CDA-mediated conversion to difluorodeoxyuridine. Experimental evidences demonstrate that CDA expression is high in gemcitabine-resistant cells^{10,11}, while tetrahydro uridine (THU), a non-specific CDA inhibitor¹², increases the sensitivity to gemcitabine¹³. Macrophages were found to mediate gemcitabine resistance of PDA by upregulating CDA in cancer cells¹⁴ and nab-Paclitaxel potentiates gemcitabine activity by reducing CDA levels in a mouse model of PDA¹⁵. Thus, there are evidences lending credence to CDA as a key protein involved in the resistance of PDA cells to treatment. Accordingly, the inventors generated CDA-null human PDA-derived cell lines using lentiviral vectors encoding specific shRNAs. The inventors found that targeting CDA strongly sensitizes PDA-derived cells to chemotherapy, both in vitro and in vivo, and induces apoptosis (data not shown).

However, the genetic depletion of CDA per se, in the absence of chemotherapy, unexpectedly inhibited PDA-derived cells proliferation, altered cell cycle progression, with a prolonged S phase, a hallmark of DNA replication stress, and impaired tumor growth, as half of the mice engrafted with Mia PACA-2-null CDA were free of tumors (FIG. 2).

There is no disclosure in the art of the role of CDA in PDA in the absence of chemotherapy, and the use of CDA inhibitors in the treatment of PDA in the absence of chemotherapy.

### SUMMARY OF THE INVENTION

The present invention relates to methods and pharmaceutical compositions for use in the treatment of pancreatic cancer in a subject in need thereof.

### DETAILED DESCRIPTION OF THE INVENTION

The inventors investigated molecular mechanisms involved in the resistance of PDA cells to treatment, the role of cytidine deaminase (CDA) in the resistance of PDA cells to treatment and in PDA in absence of treatment. From subject cohorts, the inventors identified cytidine deaminase (CDA), which catalyzes the hydrolytic deamination of cytidine and deoxycytidine to uridine and deoxyuridine, respectively, as overexpressed (i) in cohorts of subjects with PDA resisting to gemcitabine, (ii) in PDA tissue as compared to normal parenchyma, and (iii) in subjects with PDA receiving gene therapy. As expected, targeting CDA at the genetic level sensitizes cancer cells to chemotherapy (gemcitabine dFdC) both in vitro and in vivo in experimental models of PDA, with very high efficacy. To their surprise, CDA targeting in the absence of chemotherapy strongly alters cell proliferation and tumor progression, when more than half of mice engrafted with CDA-null human PDA cells remained free of tumors. Using high throughput transcriptomic, proteomic and metabolomic studies, the inventors identified massive concomitant changes in tumor cell biology following CDA ablation that can broadly be categorized into alterations of both energetic and intermediate metabolism.

Accordingly, the present invention relates to a method for treating pancreatic cancer in a subject in need thereof comprising administering to the subject a therapeutically effective amount of a cytidine deaminase inhibitor in combination with an anti-pancreatic cancer treatment selected from the group consisting of CHK1 inhibitor, WEE1 inhibitor, ART inhibitor, DHODH inhibitor or gene therapy.

As used herein, the term “subject” denotes a mammal. Typically, a subject according to the invention refers to any subject (preferably human) afflicted with pancreatic cancer. In a particular embodiment, the term “subject” refers to any subject (preferably human) afflicted with Pancreatic ductal adenocarcinoma (PDAC).

The method of the invention may be performed for any type of pancreatic cancer. The term “pancreatic cancer” refers to pancreatic cancer such as revised in the World Health Organisation Classification C25. The term “pancreatic cancer” also refers to Pancreatic ductal adenocarcinoma (PDAC) (31-35). The term “pancreatic cancer” also refers to metastatic pancreatic cancer, exocrine pancreatic cancer and locally advanced PDAC.

In some embodiments, the subject suffers from a KRAS-associated pancreatic cancer. As used herein, the term “KRAS-associated pancreatic cancer” means a cancer in which the initiation and/or maintenance are/is dependent, at least in part, on an activating mutation in a KRAS gene (also known as V-Ki-ras2 Kirsten rat sarcoma viral oncogene homolog). Typically, an “activating mutation” is one which leads to constitutive activation of the KRAS gene. Oncogenic Kras mutations include, without limitation, KrasG12D, KrasG12V, KrasG13D, KrasG12C, KrasQ61R, KrasQ61L, KrasQ61K, KrasG12R, and KrasG12C. The presence of an oncogenic Kras mutation in a sample, e.g., from a cell, tumor biopsy, or other DNA, R A or protein-containing sample can be determined at the genomic, RNA or protein level according to any suitable method known in the art.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “cytidine deaminase” and “CDA” has its general meaning in the art and refers to cytidine deaminase “CDA”, a key enzyme of the pyrimidine salvage pathway that catalyzes the hydrolytic deamination of cytidine and deoxycytidine to uridine and deoxyuridine, respectively⁹. Because of the structural similarity to cytidine, several nucleoside-based drugs are also subject to deamination by CDA (Ferraris et al, 2014). In Pancreatic ductal adenocarcinoma, cytidine deaminase “CDA” inactivates gemcitabine via CDA-mediated conversion to difluorodeoxyuridine.

The term “cytidine deaminase inhibitor” or “CDA inhibitor” has its general meaning in the art and refers to a compound that selectively blocks or inactivates the cytidine deaminase. The term “cytidine deaminase inhibitor” also refers to a compound that selectively blocks or inactivates hydrolytic deamination mediated by the cytidine deaminase. As used herein, the term “selectively blocks or inactivates” refers to a compound that preferentially binds to and blocks or inactivates CDA with a greater affinity and potency, respectively, than its interaction with the other sub-types of the deaminase family. Compounds that block or inactivate CDA, but that may also block or inactivate other deaminase sub-types, as partial or full inhibitors, are contemplated. The term “CDA inhibitor” also refers to a compound that inhibits CDA expression. Typically, a CDA inhibitor compound is a small organic molecule, a polypeptide, an aptamer, an antibody, an intra-antibody, an oligonucleotide or a ribozyme. Tests and assays for determining whether a compound is a CDA inhibitor are well known by the skilled person in the art such as described in Ferraris et al, 2014; U.S. Pat. No. 6,136,791; WO2009/052287.

CDA inhibitors are well-known in the art such as illustrated by Ferraris et al, 2014; U.S. Pat. No. 6,136,791; WO2009/052287.

In one embodiment of the invention, CDA inhibitors include but are not limited to Tetrahydrouridine (THU); Fluorinated Tetrahydrouridines and derivatives thereof such as 2′-fluorinated tetrahydrouridine derivatives;
2′-Deoxy-2′,2′-difluoro-5,6-dihydrouridine;
(4R)-2′-Deoxy-2′,2′-difluoro-3,4,5,6-tetrahydrouridine;
(4S)-2′-Deoxy-2′,2′-difluoro-3,4,5,6-tetrahydrouridine;
1-(2-Deoxy-2,2-difluoro-β-D-erythro-pentofuranosyl)-tetrahydro-2(1H)-pyrimidinone;
2′-Deoxy-2′-fluoro-5,6-dihydrouridine;
(4R)-2′-Deoxy-2′-fluoro-3,4,5,6-tetrahydrouridine;
(4S)-2′-Deoxy-2′-fluoro-3,4,5,6-tetrahydrouridine;
1-(2-Deoxy-2-fluoro-(3-D-ribofuranosyl)tetrahydro-2(1H)-pyrimidinone;
1-(2-Deoxy-2-fluoro-β-D-arabinofuranosyl)dihydro-2,4-(1H,3H)-pyrimidinedione;
(4R)-1-(2-Deoxy-2-fluoro-(3-D-arabinofuranosyl)tetrahydro-4-hydroxy-2(1H)-pyrimidinone;
(4S)-1-(2-Deoxy-2-fluoro-(3-D-arabinofuranosyl)tetrahydro-4-hydroxy-2(1H)-pyrimidinone; and compounds described in Ferraris et al, 2014.

In one embodiment of the invention, CDA inhibitors include but are not limited to difluorotetrahydrouridine derivatives; 2′-fluoro-2′-deoxytetrahydrouridines;
2′2′-DiFluoro-DiHydro-Uridine (DFDHU);
2′2′-DiFluoro-TetraHydroUridine (DFTHU);
2′(R)-fluoro-2′deoxy-tetrahydrouridines;
2′(R)-Fluoro-2′deoxy-DiHydroUridine ((R)-FDHU);
2′(S)-fluoro-2′deoxy-tetrahydrouridines;
2′(S)-fluoro-2′deoxy-dihydrouridine ((S)-FDHU);
2′(S)-fluoro-2′deoxy-tetrahydrouridine ((S)-FTHU); and compounds described in WO2009/052287.

In one embodiment of the invention, CDA inhibitors include but are not limited to ASTX727 (E7727); 5-methyl-2′,3′-dideoxy-3′-azidocytidine (5mAZC); 5-methyl-2′,3′-dideoxycytidine; 5-ethyl-2′,3′dideoxy-3′-azidocytidine; 5-propyl-2′,3′-dideoxycytidine; 5-propyl-2′,3′-dideoxy-3′-azidocytidine; 5-propene-2′,3′-dideoxy-3′-azidocytidine; 5-propyne-2′,3′-dideoxy-3′-azidocytidine; and 5-propyne-2′,3′-dideoxy-3′-azidocytidine; analogues thereof or a pharmaceutically effective salt thereof, and compounds described in U.S. Pat. No. 6,136,791; and Zebularine (1-(β-D-Ribofuranosyl)-2(1H)-pyrimidinone) (Lemaire et al., 2009; Marquez et al., 2005).

In another embodiment, the CDA inhibitor of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against CDA of the invention as above described, the skilled man in the art can easily select those blocking or inactivating CDA.

In another embodiment, the CDA inhibitor of the invention is an antibody (the term including “antibody portion”).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody. Antibodies are prepared according to conventional methodology. For instance, monoclonal antibodies may be generated using the method of Kohler and Milstein (Nature, 256:495, 1975).

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”.

In one embodiment, the CDA inhibitor of the invention is a CDA expression inhibitor. The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins (e.g., CDA) modified by, for example, methylation, acetylation, phosphorylation, ubiquitination, SUMOylation, ADP-ribosylation, myristilation, and glycosylation. Accordingly, an “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of CDA mRNA by binding thereto and thus preventing protein translation or increasing mRNA degradation, thus decreasing the level of CDA, and thus activity, in a cell. For example, antisense oligonucleotides of at least about 15 bases and complementary to unique regions of the mRNA transcript sequence encoding CDA can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. CDA gene expression can be reduced by contacting a subject or cell with a small double stranded RNA (dsRNA), or a vector or construct causing the production of a small double stranded RNA, such that CDA gene expression is specifically inhibited (i.e. RNA interference or RNAi). In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone non homologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13). Typically, the inhibitor of expression is delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing CDA. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In one embodiment, viral vectors are oncolytic viruses. Oncolytic virus refers to any virus capable of replicating in and killing tumor cells. Preferably, the virus is engineered e.g. to increase tumor cell selectivity. Representative examples of oncolytic virus include without limitation, adenovirus, reovirus, herpes simplex virus (HSV), Newcastle disease virus, poxvirus, myxoma virus, rhabdovirus, picornavirus, influenza virus, coxsackievirus and parvovirus. In preferred embodiments, the oncolytic virus is a vaccinia virus (e.g. Copenhagen, Western Reserve, Wyeth strain), rhabdovirus (e.g. vesicular stomatitis virus (VSV)), or adenovirus (e.g. ONYX-015, Delta-24-RGD). In a particularly embodiment, the oncolytic virus is an adenovirus such as Delta-24-RGD (Fueyo J et al, Oncogene, 19:2-12 (2000)). Oncolytic viruses include adenovirus, vaccinia virus, herpes virus, herpes simplex virus, reovirus, Seneca valley virus coxsackievirus, measles virus, poliovirus, VSV/rhabdovirus, parvovirus, retroviruses and viruses described in Kaufman et al., 2015; Chioccal and Rabkin, 2014.

Typically the inhibitors according to the invention as described above are administered to the subject in a therapeutically effective amount. By a “therapeutically effective amount” of the inhibitor of the present invention as above described is meant a sufficient amount of the inhibitor for treating pancreatic cancer at a reasonable benefit/risk ratio applicable to any medical treatment. It will be understood, however, that the total daily usage of the inhibitors and compositions of the present invention will be decided by the attending physician within the scope of sound medical judgment. The specific therapeutically effective dose level for any particular subject will depend upon a variety of factors including the disorder being treated and the severity of the disorder; activity of the specific inhibitor employed; the specific composition employed, the age, body weight, general health, sex and diet of the subject; the time of administration, route of administration, and rate of excretion of the specific inhibitor employed; the duration of the treatment; drugs used in combination or coincidential with the specific inhibitor employed; and like factors well known in the medical arts. For example, it is well known within the skill of the art to start doses of the inhibitor at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the inhibitor of the present invention for the symptomatic adjustment of the dosage to the subject to be treated. A medicament typically contains from about 0.01 mg to about 500 mg of the inhibitor of the present invention, preferably from 1 mg to about 100 mg of the inhibitor of the present invention. An effective amount of the drug is ordinarily supplied at a dosage level from 0.0002 mg/kg to about 20 mg/kg of body weight per day, especially from about 0.001 mg/kg to 7 mg/kg of body weight per day. In a particular embodiment, the inhibitor according to the invention may be used in a concentration between 0.01 μM and 20 μM, particularly, the inhibitor of the invention may be used in a concentration of 0.01, 0.05, 0.1, 0.2, 0.3, 0.4, 0.5, 1.0, 2.5, 3.0, 3.5, 4.0, 4.5, 5.0, 10.0, 15.0, 20.0 μM.

In a further aspect, the method of the invention comprises the step of administering the subject with the CDA inhibitor according to the invention in combination with anti-pancreatic cancer treatment.

The term “pancreatic cancer treatment” has its general meaning in the art and refers to any type of pancreatic cancer therapy undergone by the pancreatic cancer subjects including surgical resection of pancreatic cancer, and any type of anti-pancreatic cancer compound such as fluorouracil, FOLFIRINOX (fluorouracil, irinotecan, oxaliplatin, and leucovorin), nab-paclitaxel, inhibitors of programmed death 1 (PD-1), PD-1 ligand PD-L1, anti-CLA4 antibodies, EGFR inhibitors such as erlotinib, inhibitors of PARP, inhibitors of Sonic Hedgehog, gene therapy and radiotherapy.

In some embodiments, the CDA inhibitor is administered to the subject in combination with gene therapy. The term “gene therapy” denotes the therapeutic gene transfer using expression vector coding for at least one gene selected from the group consisting of SSTR2, DCK and UMK to restore gene expression. The term “gene therapy” also refers to therapeutic gene transfer using non-viral vectors to restore expression of at least one gene selected from the group consisting of SSTR2, DCK and UMK such as described in WO 2009/056434. In a preferred embodiment of the invention, the term “gene therapy” refers to delivering DCK::UMK fusion gene, encoding for both DCK and UMK, using for example non-viral vectors.

Another aspect of the present invention relates to a method for treating pancreatic cancer in a subject in need thereof comprising administering to the subject a therapeutically effective amount of a cytidine deaminase inhibitor in combination with DCK::UMK fusion gene therapy and gemcitabine.

The term “SSTR2” has its general meaning in the art and refers to somatostatin receptor subtype 2 such as described in WO 2009/056434. The term “DCK” has its general meaning in the art and refers to DeoxyCytidine Kinase such as described in WO 2009/056434. The term “UMK” has its general meaning in the art and refers to Uridylate Monophosphate Kinase such as described in WO 2009/056434.

In some embodiments, the CDA inhibitor is administered to the subject in combination with a CHK1 inhibitor. As used herein, the term “CHK1 inhibitor” means any agent, whether now known or after-discovered, whether naturally occurring, isolated from nature, or man-made, that is capable of at least partially abrogating cell cycle checkpoint activity of the checkpoint kinase 1, commonly referred to as CHK1. Such agents can be alternatively referred to as “capable of inhibiting CHK1.” Such agents include, but are not limited to, small molecule compounds, biologies, and antisense agents. One specific example of a class of CHK1 inhibitors is a disubstituted urea compound, wherein each nitrogen of the urea compound is substituted with one or more aryl and/or heteroaryl moieties. CHK1 inhibitors are known in the art, for example, the CHK1 inhibitor can include, for example, a peptide, an antibody, an antisense molecule or a small molecule. CHK1 inhibitors useful in the present invention include but are not limited to, those described or claimed in the following publications the entire disclosures of which are incorporated by reference herein. Compounds useful in the present invention as CHK1 inhibitors include, but are not limited to, disubstituted ureas. Disubstituted ureas, as used herein, refers to urea compounds having one substituent at each nitrogen (N and N′) wherein each substituent is optionally substituted and selected from the group consisting of aryl, heteroaryl, cycloalkyl, heterocycloalkyl, and Ci_6 alkyl substituted with a heteroaryl or aryl moiety. In some embodiments, the urea compounds comprise two aryl substituents, while in other embodiments, the urea compounds comprise one aryl and one heteroaryl substituent, and in still other embodiment, the urea compounds comprise two heteroaryl substituents. Specific examples of disubstituted ureas include, but are not limited to, those described or claimed in the following publications, the entire disclosures of which are incorporated herein by reference: aryl and heteroaryl substituted urea compounds described in any one of the following patents and patent applications: U.S. Pat. No. 7,067,506; WO 2002/070494; WO 06/012308; WO 06/014359; WO 06/021002; International Patent Application No. PCT/US06/11584; and U.S. Provisional Patent Application No. 60/818,008, filed Jun. 30, 2006; diaryl urea compounds described in U.S. Patent Publication No. 2004/0014765; diaryl urea compounds described in WO 03/101444; diaryl urea compounds described in U.S. Pat. No. 7,056,925 and WO 04/014876; disubstituted urea compounds described in WO 06/0725891; and macrocyclic disubstituted urea compounds described in WO 05/215556. Other compounds useful as CHK1 inhibitors include methylxanthines and related compounds (Fan et al, Cancer Res. 55: 1649 (1995)); ureidothiophenes (WO 03/029241 and WO 03/028731); N-pyrrolopyridinyl carboxamides (WO 03/028724); antisense CHK1 oligonucleotides (WO 01/57206 and U.S. Pat. No. 6,211,164); genes which modulate CHK1 (WO 04/004785); CHK1 receptor antagonists (WO 00/16781); heteroaromatic carboxamide derivatives (WO 03/037886); aminothiophenes (WO 03/029242); (indazolyl) benzimidazoles (WO 03/004488); benzimidazole quinolinones (U.S. Patent Publication No. 2004/0092535 and WO 04/018419); heterocyclic-hydroxyimino-fluorenes (WO 02/16326); scytoneman skeleton containing derivatives (scytonemin) (U.S. Pat. No. 6,495,586); heteroarylbenzamides (WO 01/53274); indazole compounds (WO 01/53268); indolacarbazoles (Tenzer et al., supra); chromane derivatives (WO 02/070515); paullones (Schultz, et al., J. Med. Chem., 42:2909 (1999)); indenopyrazoles (WO 99/17769); flavones (Sedlacek et al., Int. J. Oncol. 9: 1143 (1996)); peptide derivatives of peptide loop of serine threonine kinases (WO 98/53050); oxindoles (WO 03/051838); diazepinoindolones (WO 04/063198); pyrimidines (WO 04/048343); pyrrolocarbazoles, benzofuroisoindoles, and azacyclopentafluorenes (WO 03/091255); fused pyrazoles (WO 06/074281); naphthyridines (WO 02/100356); aryl carbonyl derivatives (WO 04/002481); substituted pyridine derivatives (WO 03/093297); pyrimidin-4-yl-1H-indazol-5-yl-amines (WO 05/103036); substituted thiophenes (WO 05/16909); substituted pyran derivatives (WO 04/97426), pyrazolopyrimidine derivatives (WO 04/87707); aminopyrazole derivatives (WO 05/9435); and fused pyrazoles (WO 06/74281 and WO 06/74207), each incorporated herein by reference.

In some embodiments, the CDA inhibitor is administered to the subject in combination with a WEE1 inhibitor. Wee1-like protein kinase (“WEE1”) is a tyrosine kinase. WEE1 is inactivated in normal cells through phosphorylation and degradation during the M phase. WEE1 negatively regulates entry into mitosis by phosphorylating Cdc2 (Stathis, Anastaslos and Amit Oza, “Targeting Wee1-like Protein Kinase To Treat Cancer.” Drug News & Perspectives. 23(7) (2010) 425-429). Entry into mitosis is triggered by CDC25, which dephosphorylates Cdc2. WEE1 inhibitors are known, see for example, International Publication WO 2010/098367, International Publication WO 2010/067886, International Publication WO 2008/115742, International Publication WO 2008/115738, International Publication WO 2007/126122, International Publication WO 2007/126128, International Publication WO 2004/007499 and United States Patent Application Publication 2005/0037476. WEE1 inhibitors include MK-1775, PD-166285 (also known as PD0166285) and PF-00120130.

In some embodiments, the CDA inhibitor is administered to the subject in combination with an inhibitor of DHODH. As used herein the term “dihydroorotate dehydrogenase (DHODH)” refers to the enzyme that catalyzes the fourth step in the de novo biosynthesis of pyrimidine. DHODH converts dihydroorotate to orotate. Any inhibitor of DHODH can be used in methods of the invention. As used herein, the phrase “inhibitor of DHODH” means a compound or agent that inhibits the biological activity of DHODH. The biological activity of DHODH can be inhibited using a compound or agent that inhibits the enzymatic activity of DHODH, or a compound or agent that down regulates expression or availability of DHODH in a cell or organism (e.g. siRNA, shRNA). Many inhibitors of DHODH are known to those skilled in the art. For example, various inhibitors are described in: Leban et al. (2005) SAR, species specificity, and cellular activity of cyclopentene dicarboxylic acid amides as DHODH inhibitors, Bioorganic & Medicinal Chemistry Letters 15(21): 4854-4857; and Fritzson et al. (2010) Inhibition of human DHODH by 4-Hydroxycoumarins, fenamic acids, and N-(alkylcarbonyl)anthranilic acids identified by structure-guided fragment selection Chem Med Chem 5(4): 608-617; Kulkarni et al. (2010) 4SC-101 a novel small molecule dihydroorotate dehydrogenase inhibitor, suppresses systemic lupus erythematosus in MRL-(Fas)lpr mice Am J Pathol. 176(6):2840-7. Various DHODH inhibitors have been disclosed. See for example WO2010083975; WO2011138665; WO200137081; WO2009133379; WO 2009021696; WO200 082691; WO2009029473; WO2009153043; US2009209557; US2009 062318; US2009082374; WO2008097180; W02QQ8Q77639; US2008027079; US2007 299U4; US2007027193; US2007224672; WO2007149211; JP2007015952; WO2006 044741; WO2006001961; WO2006051937; WO2006038606; WO2006022442; US2006 199856; WO2005075410; U.S. Pat. No. 7,074,831; WO2004056797; U.S. Pat. No. 7,247,736; WO2004056747; WO 2004056746; JP2004099586; WO2003097574; WO2003030905; WO2003006425; WO2003 006424; US2003203951; WO2002080897; U.S. Pat. No. 7,176,241; 7,423,057; WO2001024785; U.S. Pat. No. 6,841,561; WO9945926; WO9938846; WO9941239; EP767167 and U.S. Pat. No. 5,976,848, each of which are herein incorporated by reference in their entirety. For additional reviews and literature regarding DHODH inhibitors see Bio & Med. Chem. Letters, 20(6), 2010, pages 1981-1984; Med. Chem. 2009, 52, 2683-2693; J. Med. Chem. 2008, 51 (12), 3649-3653. For example, inhibitors include, but are not limited to, leflunomide, teriflunomide, brequinar (NSC 368390) (Cancer Research 1992, 52, 3521-3527), Dichloroallyl lawsone (The Journal of Biological Chemistry 1986, 261(32), 14891-14895; McKelvey et al. Clin Pharmacol Ther. 1979 May; 25(5 Pt 1):586-90.), Maritimus (F 778) (Drugs of the Future 2002, 27(8), 733-739) and Redoxal (The Journal of Biological Chemistry 2002, 277(44), 41827-41834). See also Example 1 and FIG. 5, which describes a compound of previously unknown function, NSC210627, with similarity to brequinar, a known inhibitor of DHODH.

In some embodiments, the CDA inhibitor is administered to the subject with an ATR inhibitor. As used herein, the term “ATR” kinase refers to ataxia telangiectasia and Rad3 related kinase. As used herein, the term “ATR inhibitor” refers to a composition or compound that inhibits ATM activity, either directly or indirectly, using any method known to the skilled artisan. An ATM inhibitor may be any type of compound, including but not limited to, a nucleic acid, peptide, antibody, small molecule, antagonist, aptamer, or peptidomimetic.

Example of ATR inhibitors include but are not limited to Schisandrin B (10.Benzo(3,4)cycloocta(1,2-f)(1,3)benzodioxole, 5,6,7,8-tetrahydro-1,2,3,13-tetramethoxy-6,7-dimethyl-, stereoisomer: NU6027 (6-(cyclohexylmethoxy)-5-nitrosopyrimidine-2,4-diamine); NVP-BEZ235 (2-methyl-2-[4-(3-methyl-2-oxo-8-quinolin-3-ylimidazo [4,5-c] quinolin-1-yl)phenyl]propanenitrile); VE-821 (2-(aminomethyl)-6-[4,6-diamino-3-[4-amino-3,5-dihydroxy-6-(hydroxymethyl)oxan-2-yl]oxy-2-hydroxycyclohexyl]oxyoxane-3,4,5-triol; sulfuric acid); VE-822 (VX-970; 3-[3-[4-(methylaminomethyl)phenyl]-1,2-oxazol-5-yl]-5-(4-propan-2-ylsulfonylphenyl)pyrazin-2-amine); AZ20 ((3R)-4-[2-(3H-indol-4-yl)-6-(1-methylsulfonylcyclopropyl)pyrimidin-4-yl]-3-methylmoφholine); AZD6738 (4-[4-[1-[[S(R)]-S-methylsulfonimidoyl]cyclopropyl]-6-[(3R)-3-methyl-4-mopholinyl]-2-pyrimidinyl]-1H-pyrrolo[2,3-b]pyridine); and ETP-46464 (2-methyl-2-[4-(2-oxo-9-quinolin-3-yl-4H-[1,3]oxazino[5,4-c]quinolin-1-yl)phenyl]propanenitrile); see, e.g., Weber and Ryan, Pharmacology & Therapeutics 149: 124-138 (2015); Toledo et al., Nat Struct Mol Biol. 2011 June; 18(6):721-7; US20140356456; and US20100048923.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is at least a CDA inhibitor. In some instance, the combination of the present invention induces the synthetic lethality of the cancer cells.

In a further aspect, the present invention relates to a method of screening a candidate compound for use as a drug for treating pancreatic cancer in a subject in need thereof, wherein the method comprises the steps of:
providing a CDA, providing a cell, tissue sample or organism expressing a CDA,
providing a candidate compound such as a small organic molecule, a polypeptide, an aptamer, an antibody or an intra-antibody,
measuring the CDA activity,
and selecting positively candidate compounds that inhibit CDA activity.

Methods for measuring CDA activity are well known in the art (Ferraris et al., 2014). For example, measuring the CDA activity involves determining a Ki on the CDA cloned and transfected in a stable manner into a CHO cell line and human recombinant CDA, measuring CDA catalysed deamination of CDA substrates such as cytidine to uridine in the present or absence of the candidate compound. Tests and assays for screening and determining whether a candidate compound is a CDA inhibitor are well known in the art (Ferraris et al., 2014). In vitro and in vivo assays may be used to assess the potency and selectivity of the candidate compounds to inhibit CDA activity. Activities of the candidate compounds, their ability to bind CDA and their ability to inhibit CDA activity may be tested using CDA assay such as described for example in Ferraris et al., 2014, isolated pancreatic cancer cells or CHO cell line cloned and transfected in a stable manner by the human CDA or methods such as described in the Example. Activities of the candidate compounds and their ability to bind to the CDA, or their ability to inhibit CDA activity may be assessed by the determination of a Ki on the CDA cloned and transfected in a stable manner into a CHO cell line, human recombinant CDA, measuring pancreatic cancer cells apoptosis induction, pancreatic cancer cells proliferation inhibition, alteration of pancreatic cancer cell cycle progression, tumor growth inhibition in the present or absence of the candidate compound. The ability of the candidate compounds to inhibit CDA activity may be assessed by measuring CDA catalysed deamination of CDA substrates, total ATP level, mitochondrial ROS production, and reduced GSH/oxidized GSSG such as described in the example. Cells expressing another deaminase than CDA or mutated CDA may be used to assess selectivity of the candidate compounds.

The inhibitors of the invention may be used or prepared in a pharmaceutical composition. Typically, the inhibitor of the invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, intramuscular, intravenous, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, intraperitoneal, intramuscular, intravenous and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active inhibitors in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject. In addition to the inhibitors of the invention formulated for parenteral administration, such as intravenous or intramuscular injection, other pharmaceutically acceptable forms include, e.g. tablets or other solids for oral administration; liposomal formulations; time release capsules; and any other form currently used. Pharmaceutical compositions of the invention may include any further compound which is used in the treatment of pancreatic cancer. In one embodiment, said additional active compounds may be contained in the same composition or administrated separately.

In another embodiment, the pharmaceutical composition of the invention relates to combined preparation for simultaneous, separate or sequential use in treating pancreatic cancer in a subject in need thereof. The invention also provides kits comprising the inhibitor of the invention. Kits containing the inhibitor of the invention find use in therapeutic methods.

The invention will be further illustrated by the following figures and examples. However, these examples and figures should not be interpreted in any way as limiting the scope of the present invention.

### FIGURES

FIG. 1. (A) CDA is overexpressed in tumors (n=20, matched pairs), (B) in subjects treated with gemcitabine (n=50) and (C) in subjects receiving gene therapy (n=14).

FIG. 2. CDA targeting alters cell cycle (A) induces cell death by apoptosis (B), inhibits cell proliferation (C) and tumor growth (D) in the absence of chemotherapy.

FIG. 3. CDA targeting ATP production (A) inhibits oxygen consumption (B), induces cellular ROS (C) and decreases GSH/GSSG ratio (D) in human PDA cells.

FIG. 4. CDA targeting alters purine and PRPP synthesis (A) pyrimidine synthesis (B); and TCA cycle (C), in human PDA cells.

FIG. 5. CDA mRNA expression in normal pancreatic cells expressing hTERT, HPV E6/E7, SV40 small antigen and oncogenic KRAS.

FIG. 6: Pharmacological inhibition of CDA (A) sensitizes pancreatic cancer cells to gemcitabine but fails to alter cell proliferation (B) and tumor metabolism (C). THU: Tetrahydrouridine, DR: 1,3-diazepinone riboside.

FIG. 7: Combination of CDA targeting with glutamines synthase (CB839, A) or glycolysis (2-deoxy Glucose, B) strongly inhibits cancer cell proliferation.

### EXAMPLE 1

Pancreatic ductal carcinoma (PDA) is the most common type of pancreatic cancer Despite decades of intense efforts from researchers and clinicians, PDA remains a challenge to treat, with 5 year rates survival lower than 6% for subjects with cancers of all stages. Most PDA is identified at a late stage, when surgical intervention is not possible. Even with complete resection and negative results from analyses of tumour margins, long-term survival after surgery is poor; tumors recur in virtually all subjects. To put this into perspective, PDA is estimated to become one of the top three leading cause of cancer-related death by 2030².

Progress in the treatment of PDA has been incremental. Arguably, combination cytotoxic therapies such as FOLFIRINOX³, along with gemcitabine⁴ and albumin-bound paclitaxel⁵, have provided meaningful gains, but there is lot of needs for improvement. The only targeted agents approved in the treatment of PDA is the EGFR inhibitor Erlotinib (Tarceva), which given in combination with gemcitabine, only slightly increases overall survival time compared with gemcitabine alone⁶. Taken together, the current treatment approaches for PDA increase survival times of subjects in weeks to months.

In this dismal context, we have elected cancer gene therapy as a promising approach for PDA management⁷. We conducted the first-in-human clinical trial, based on the use of non-viral vectors to transfer anticancer genes that sensitize PDA to gemcitabine⁸. This early phase clinical trial performed in Toulouse (collaboration IUCT-CRCT Team 1) demonstrates that intratumoral gene delivery is safe and feasible in subjects with unresectable PDA. In addition, a population of subjects with locally advanced tumors benefited from this treatment, with two subjects surviving for up to two years following gene therapy⁸. A phase II clinical trial is underway.

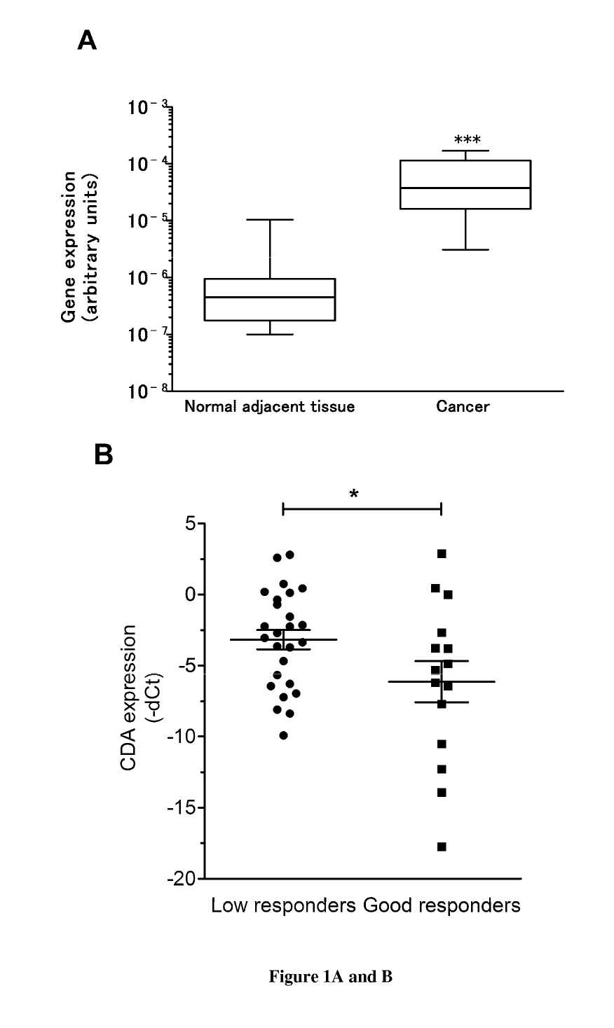

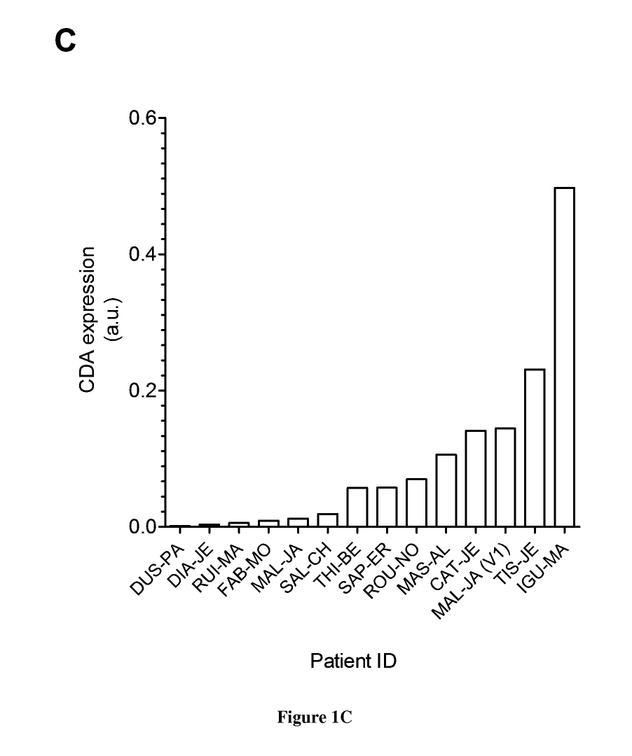

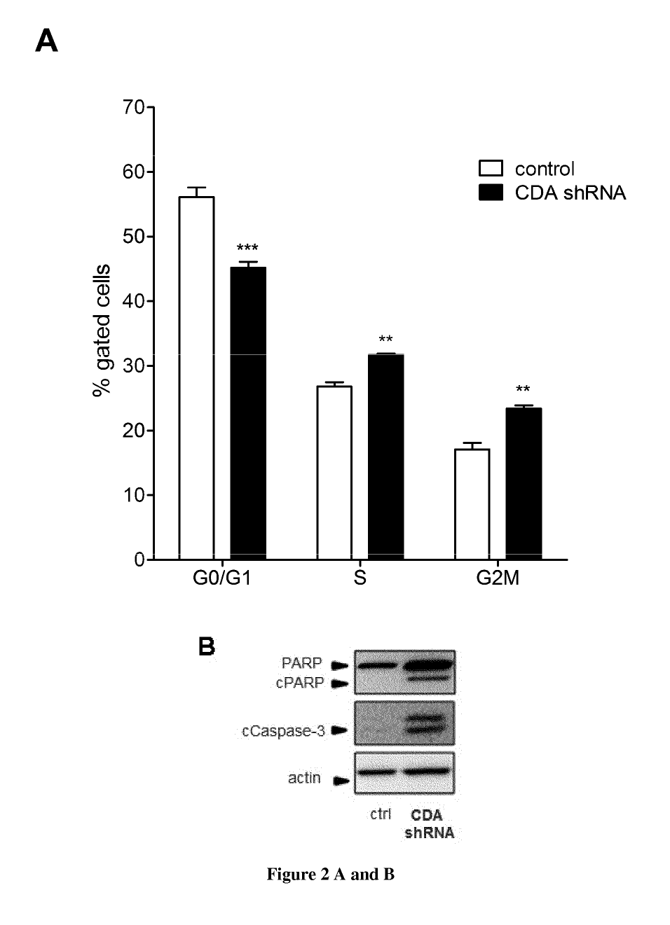

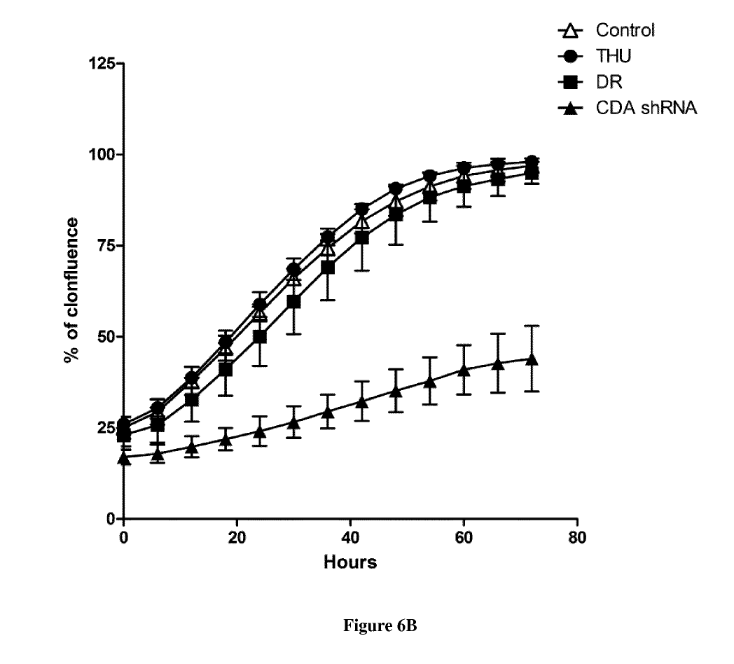

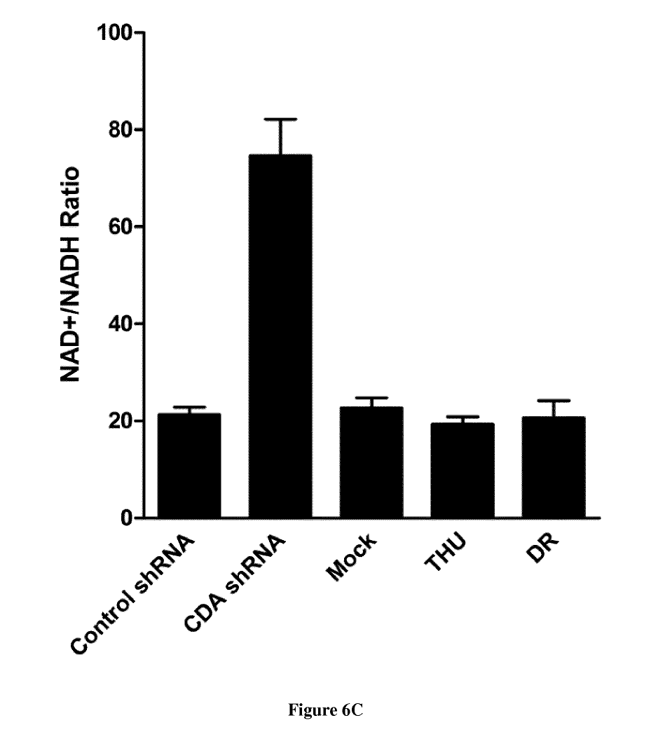

While groundbreaking, this trial also highlights the need to further characterize the molecular mechanisms involved in the resistance to treatment. Accordingly, we have interrogated our clinical samples for the expression of key proteins involved in resistance of cancer cells to gemcitabine. We found that cytidine deaminase (CDA) was the only gene (i) upregulated in resected PDA samples compared to normal parenchyma, (ii) overexpressed in microbiopsies from locally advanced and metastatic PDA resisting to therapy and (iii) detectable in microbiopsies of PDA subjects treated by gene therapy (FIG. 1). CDA is a key enzyme of the pyrimidine salvage pathway that catalyzes the hydrolytic deamination of cytidine and deoxycytidine to uridine and deoxyuridine, respectively⁹. In PDA, gemcitabine is inactivated primarily by CDA-mediated conversion to difluorodeoxyuridine. Experimental evidences demonstrate that CDA expression is high in gemcitabine-resistant cells^{10,11}, while tetrahydro uridine (THU), a non-specific CDA inhibitor¹², increases the sensitivity to gemcitabine¹³. Macrophages were found to mediate gemcitabine resistance of PDA by upregulating CDA in cancer cells¹⁴ and nab-Paclitaxel potentiates gemcitabine activity by reducing CDA levels in a mouse model of PDA¹⁵. Thus, there are mounting evidences lending credence to CDA as a key protein involved in the resistance of PDA cells to treatment. Accordingly, we generated CDA-null human PDA-derived cell lines using lentiviral vectors encoding specific shRNAs, and we found that targeting CDA strongly sensitizes PDA-derived cells to chemotherapy, both in vitro and in vivo, and induces apoptosis (data not shown). We obtained similar results in vitro in combining CDA inhibitors with chemotherapy (FIG. 6 A).

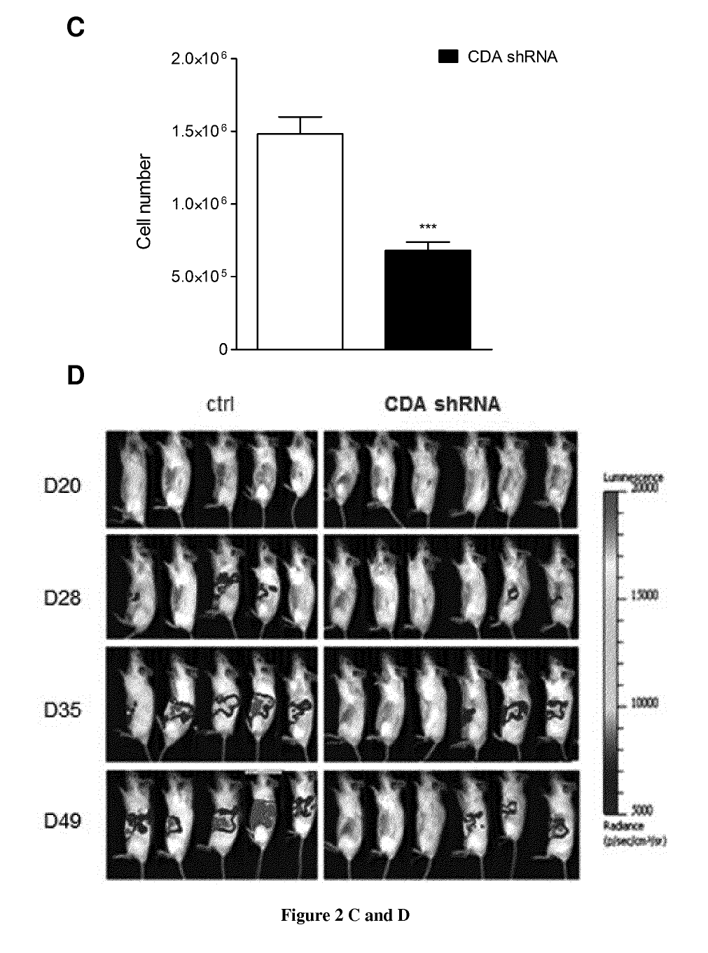

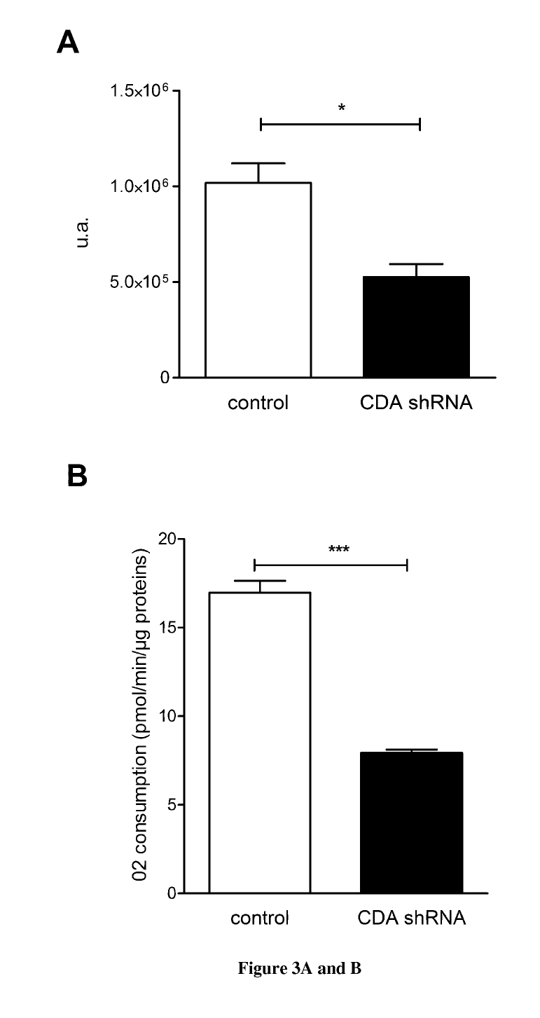

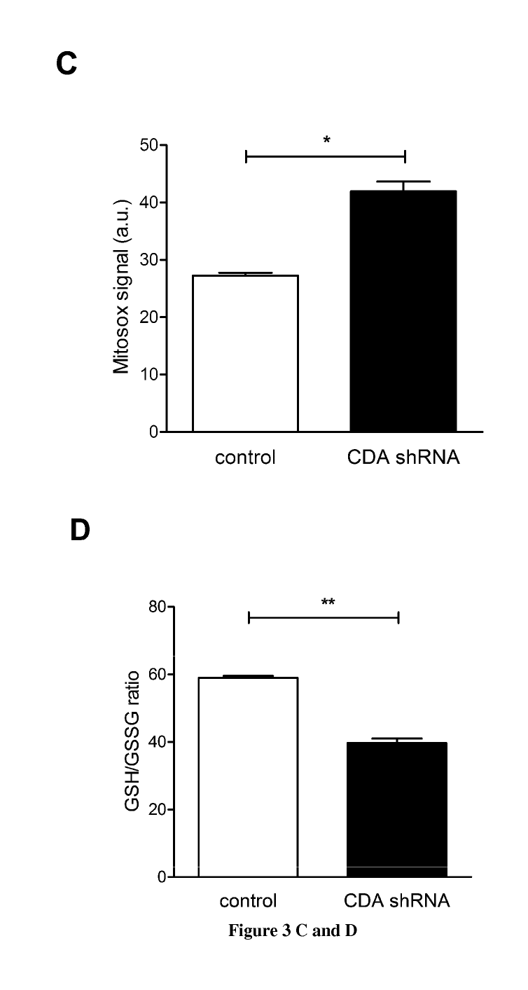

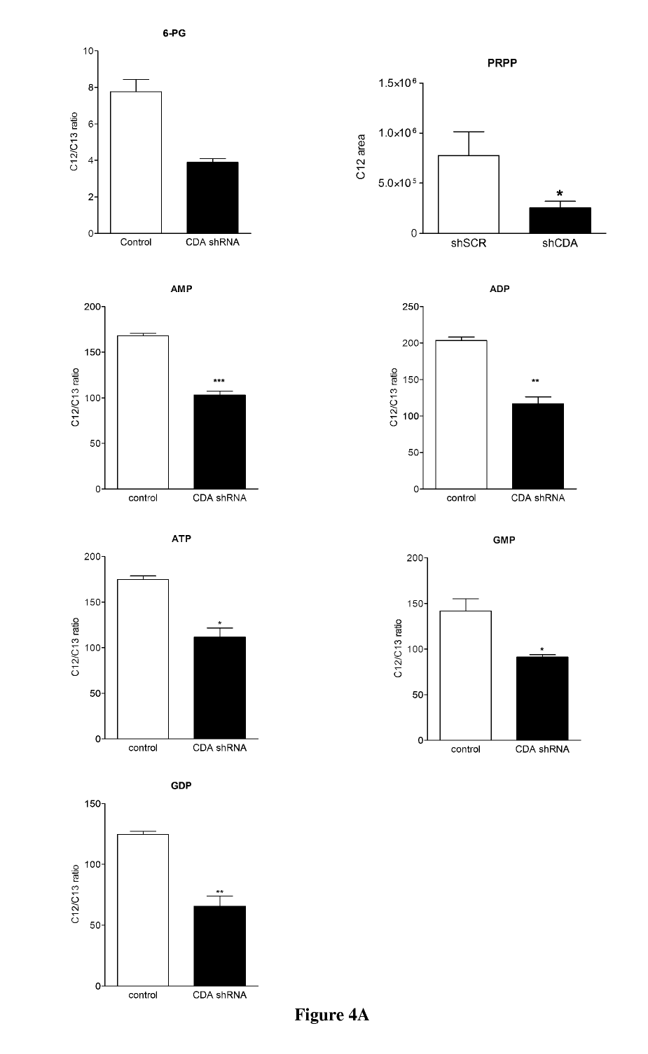

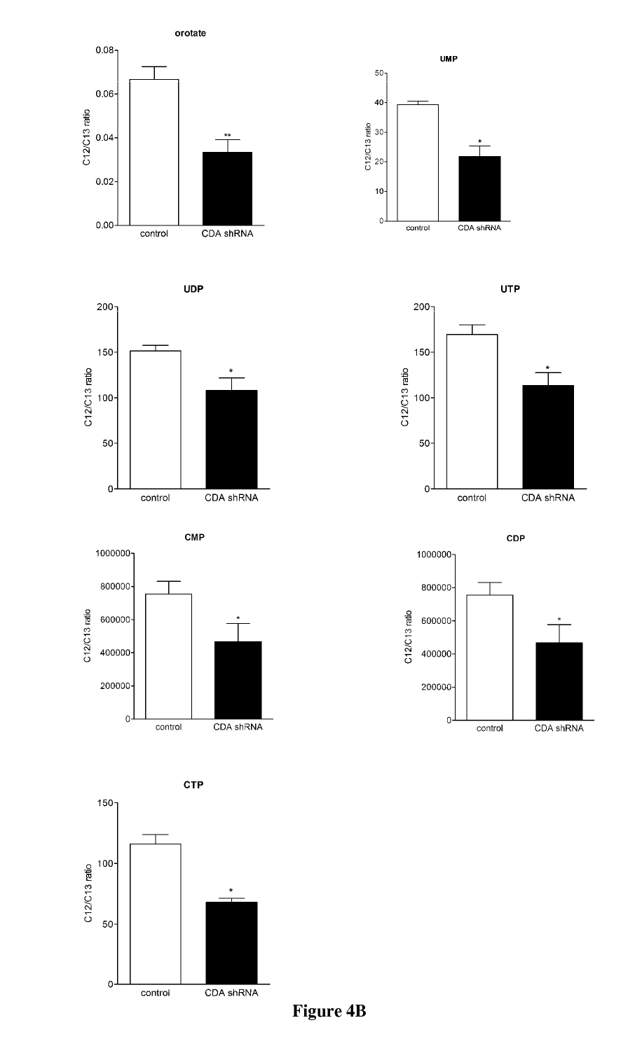

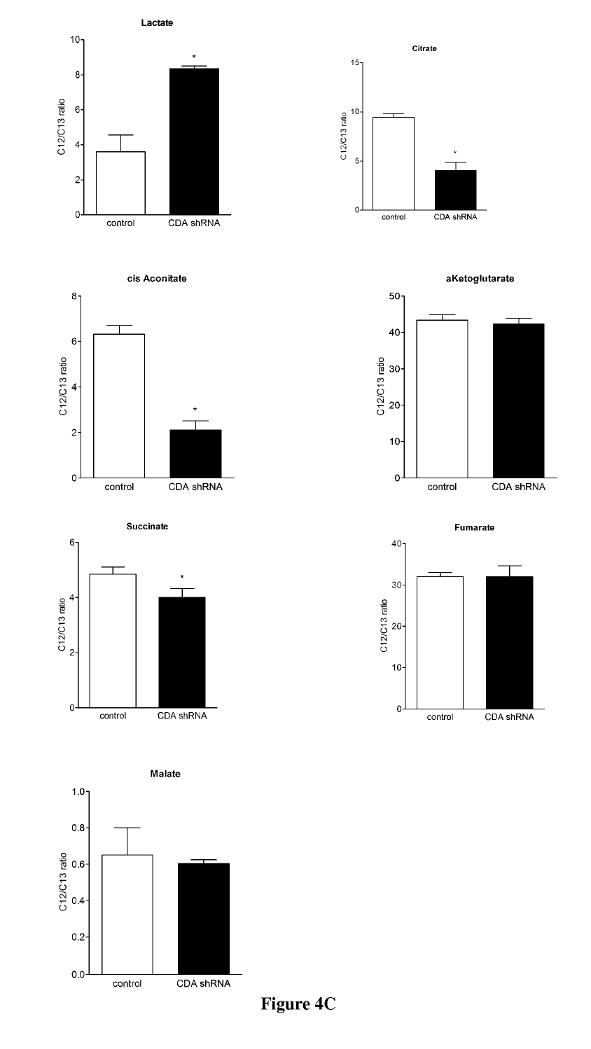

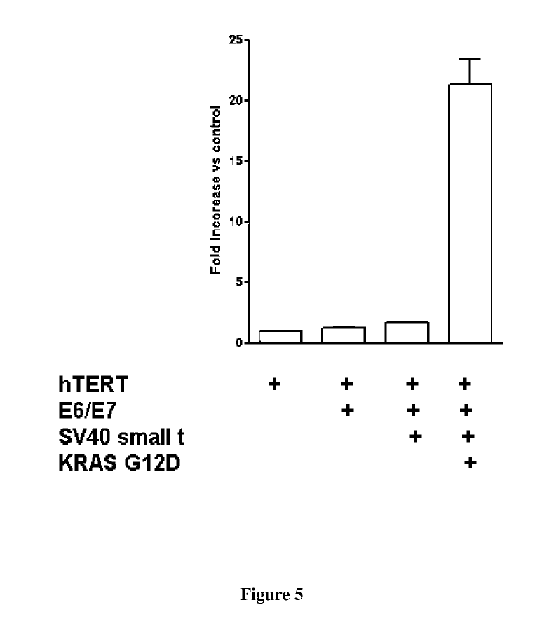

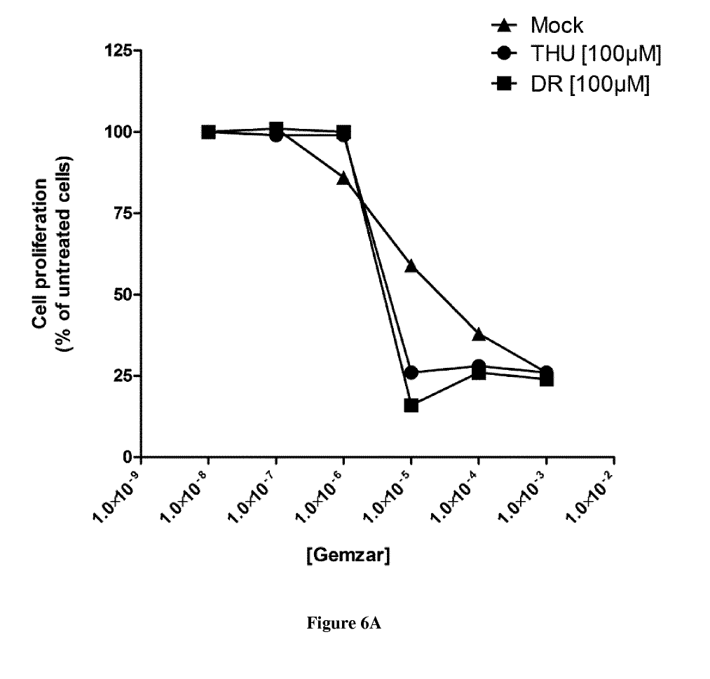

However, to our surprise, the genetic depletion of CDA per se, in the absence of chemotherapy, profoundly inhibited PDA-derived cells proliferation, altered cell cycle progression, with a prolonged S phase, a hallmark of DNA replication stress, and impaired tumor growth, as half of the mice engrafted with Mia PACA-2-null CDA were free of tumors (FIG. 2). Interestingly, the inhibition of CDA activity failed to phenocopy the consequences of the genetic targeting of the enzyme (FIG. 6 B). In order to identify the key pathways altered by CDA deficiency in PDA cells, we then performed unbiased 2D-DIGE proteomic studies. We found that the expression/stability of a number of proteins involved in mitochondrial function was altered in response to CDA deficiency. Accordingly, we identified a decrease of the total ATP level and a switch in ATP production from mitochondrial to glycolytic source, so-called Pasteur effect, as we observed when inhibiting mitochondrial oxidative phosphorylation with hypoxia or mitochondrial inhibitors e.g. rotenone, antimycin A or metformin in human glioblastoma and acute myeloid cells^{16,17}. In addition, mitochondrial oxygen consumption in CDA-depleted cells is decreased compared to controls, with elevated mitochondrial ROS production and decreased reduced GSH/oxidized GSSG, suggesting an impairment of the redox balance leading to an oxidative stress (FIG. 3). Finally, using a global LC/MS-based metabolomic approach, CDA deficiency resulted in a profound decrease in both purine and pyrimidine productions, as well as multiple alterations in TCA cycle with elevated lactate production (FIG. 4).

The aforementioned results demonstrate for the first time that targeting CDA, an enzyme involved in pyrimidine salvage pathway, strongly alters PDA proliferation and tumor progression, via massive modification of cancer cell metabolism and alteration of cell cycle, in the absence of chemotherapy.

### EXAMPLE 2

As described above, we found that PDA tumors expressed high levels of CDA compared to normal parenchyma and that targeting CDA in human PDA-derived cells strongly alters cell proliferation and tumor progression. Invasive PDA arises through multistage genetic and histologic progression from microscopic precursor lesions designated as pancreatic intraepithelial neoplasia (PanIN) that are believed to develop and progress asymptomatically over several decades^{18,19}. An early event during malignant transformation is the acquisition of activating mutations in the KRAS oncogene which occurs in >90% of subjects with PDA²⁰. PDA are highly “addicted” to this oncogene for multiple parameters influencing tumour initiation, progression and maintenance as demonstrated using genetically engineered mouse (GEM) models²¹. We obtained results showing that CDA is upregulated at the mRNA level in human pancreatic cells transformed by the KRAS oncogene (FIG. 6). We obtained preliminary data strongly suggesting that CDA is essential for PDA tumor initiation (FIG. 2).

### EXAMPLE 3

Our results demonstrate that targeting CDA in PDA cells results in alterations in both S-phase progression and energetic metabolism.

Rapid and accurate DNA replication is critical for cell proliferation and for the faithful transmission of genetic information to daughter cells. Proliferating cells are continuously exposed to a variety of events impeding the progression of replication forks, commonly referred to as replication stress (RS). Sources of RS include DNA lesions of endogenous or exogenous origin and regions of the genome that are intrinsically difficult to replicate, such as highly-transcribed genes, secondary DNA structures and tightly-bound protein-DNA complexes^{40,41}. Arrested replication forks represent a major source of genomic instability and RS has been implicated in various aspects of the cancer process. Cells exposed to acute RS activate a DNA-Damage-Response (DRR) when the fork collapse, and an ATR/Chk1-dependent checkpoint pathway known as the DNA replication checkpoint⁴² to arrest the cell cycle, limit fork collapse and delay origin firing until RS is relieved. Moreover, it coordinates fork repair processes and allows the completion of DNA replication^{43,44}

We have found that CDA depletion perturbs the dynamics of the S-phase in PDA cells (FIG. 3) and induces the phosphorylation of CHK1 kinase, two hallmarks of oncogene-driven replicative stress. This is highly unusual in the context of KRAS-promoted malignancies that commonly do not show any evidences of replicative stress nor activated DNA damage repair pathways. For instance, CHK1 inhibitors fail to hamper tumor progression in experimental models of PDA⁴⁵. In addition, we found that CDA depletion results in the inhibition of both purine and pyrimidine cellular pools (FIG. 4). Recent reports also demonstrate that CDA deficiency leads to under replication of cellular DNA, due to the partial inhibition of activity of the DNA Repair enzyme PARP-1⁴⁶.

To fuel its elevated demand for energy and macromolecular biosynthesis, PDA show increased nutrient acquisition that is coupled to increased flux through downstream metabolic pathways⁵³. In PDA cells grown in vitro, glycolysis predominates over mitochondrial oxidative phosphorylation of pyruvate, regardless of oxygen tension (Warburg effect). In PDA, oncogenic KRAS plays a vital role in controlling tumor metabolism through stimulation of glucose uptake and channeling of glucose intermediates into the hexosamine biosynthesis (HBP) and nonoxidative pentose phosphate pathways (PPP), thereby decoupling ribose biogenesis from NADP/NADPH-mediated redox control⁵⁴. Furthermore KRAS signaling drives increased glutamine metabolism for cytosolic NADPH production to maintain redox homeostasis and support cell division and proliferation in PDA cells. Accordingly, recent reports have shown preclinical and clinical relevance for targeting cell metabolism and especially glutamine metabolism to overcome chemoresistance or radioresistance in human PDA or NSCLC cancers, respectively. In CDA-null Mia PACA-2 cells, we found that global ATP levels are severely curtailed, with glycolysis and mitochondria equally participating to ATP production as a consequence of the Pasteur effect induction (FIG. 3). This massive drop in ATP levels, combined with the decrease of 6-phosphogluconate production, could account for the major impact on phosphoribosyl pyrophosphate (PRPP) production, resulting in the reduction of both purine and pyrimidine levels and cell cycle progression as well as in the induction of an energetic crisis in these cells.

Interestingly, CDA depletion does not perfectly mimics KRAS extinction leading to decrease glycolysis and lactate production⁵⁴, as fructose-1,6-diphosphate, glycerate 2/3 phosphate and lactate are elevated following CDA deficiency (FIG. 4). Accordingly, we measure the glycolytic flux in CDA-deficient cells and the expression of key proteins involved in glycolysis in response to CDA targeting. We also found that CDA inactivation is accompanied by significant alterations to TCA cycle intermediates (citrate, cis-aconitate, FIG. 4), that could be explained by the decrease of pyruvate dehydrogenase (PDH) expression identified by 2D-DIGE in CDA-null cells. However, the levels α-ketoglutarate (α-KG), succinate, fumarate and malate are maintained in these cells, suggesting alternative anaplerotic pathways for TCA supply and the absence of glutamine metabolism as opposed to KRAS extinction. In addition, the NAD+/NADH ratio, an indicator of oxidative stress buffering capacity and of biomass production by cancer cells, is severely altered in cells depleted for CDA (FIG. 6C). Again, the inhibition of CDA activity failed to phenocopy the consequences of the genetic targeting of the enzyme (FIG. 6 C). As NAD+ is an important co-factor of PARP-related tumor growth, these results give a novel opportunity of using PARPi in combination with CDA depletion. Interestingly, KRAS-driven PDA cells are known to utilize alternative carbon sources to fuel the TCA cycle, such as glutamine⁵⁴. The first step in glutamine catabolism involves its conversion to glutamate catalyzed via the glutaminase enzymes (GLS1 and GLS2). Glutamate is a source of α-KG generated by the function of glutamine dehydrogenase (GLUD1) or glutamate-oxaloacetate transaminase (GOT1, GOT2). Thus, the glutamine conversion to α-KG, oxaloacetate and aspartate could be essential to CDA-depleted cells to survive. Interestingly, we identified by 2D-DIGE in CDA-null cells a decrease of one of the SDH subunit linked to O₂ consumption decrease (FIG. 3).

Beside this, the pentose phosphate pathway (PPP) is important for tumorigenesis as it provides NADPH for macromolecules biosynthesis and ROS detoxification, as well as ribose 5-phosphate for DNA/RNA synthesis. RAS oncogene is known to promote resistance to oxidative stress trough GSH-based ROS scavenger pathways⁵⁵. Notably, we found that the oxidative phase of the PPP was impaired in CDA-null cells (as measured following 6-phosphogluconate production), while PPP non-oxidative phase is unchanged.

We state that CDA, which is upregulated in cancer, is a regulatory master of PDA oncogenesis that shares common features with KRAS oncogene but also presents unique opportunities to define new vulnerabilities for PDA treatment. Accordingly, we define opportunity to replicative stress- and metabolic-based synthetic lethality strategies for PDA tumors depleted from CDA.

### EXAMPLE 4

We have already identified that CDA depletion induces the activation of CHK-1 that is highly evocative of replicative stress in PDA cells. Indeed, The ATR-CHK1 signaling cascade has a crucial role in limiting replicative stress and can be targeted by specific inhibitors such as the Chk1 inhibitor SCH900776, to improve the effect of CDA inhibitors for their anti-proliferative and anti tumoral activities on PDA. As CHK1 activity is strongly enhanced by ATR-mediated phosphorylation, inhibition of ATR could produce similar responses to those observed with inhibition of CHK1, but with “added value”. Indeed, ATR phosphorylates SMARCAL1 (a SWI/SNF family member that has annealing helicase activity), thereby limiting its fork regression activity and preventing the collapse of stalled replication forks⁴². Accordingly, use of specific ATR inhibitors, such as VE-821 could represent an interesting combination treatment. Last, an alternative approach to infer with CHK1 is to target WEE1. This kinase phosphorylates CDK1 and CDK2, rendering them less active. When WEE1 is inhibited by drugs, CDK activity is enhanced and cells in S phase can be induced to enter mitosis prematurely, even before DNA replication is complete⁵⁶. Combined by the shortage of nucleotides provoked by CDA targeting, WEE1 inhibition could not only reduce replication fork speed but also facilitate double strand breaks. Interestingly, WEE1 inhibitors are currently under clinical evaluation for PDA. We reveal alterations in the expression/activity of alternative DNA polymerases that could also be targeted together with CDA to maximize the replicative stress in PDA cells.

### EXAMPLE 5

We have accumulated evidences that the tumor cell metabolism is massively altered following CDA targeting in PDA cells. This study reveals for the first time how essential pyrimidine salvaging is for this tumor type. Interestingly, other pathways are crucial for maintaining the cellular pyrimidine pool, such as the “de novo” pathway driven by dihydroorotate dehydrogenase (DHODH). This pathway is present and functional, it may represent another therapeutic angle to target the newly described “addiction” of PDA cells to pyrimidine. Accordingly, we evaluate the antiproliferative and antitumoral activity of leflunomide, a well-known inhibitor of DHODH, used in active moderate-to-severe rheumatoid arthritis and psoriatic arthritis⁴⁹, in combination with CDA inhibitors, in experimental models of PDA.

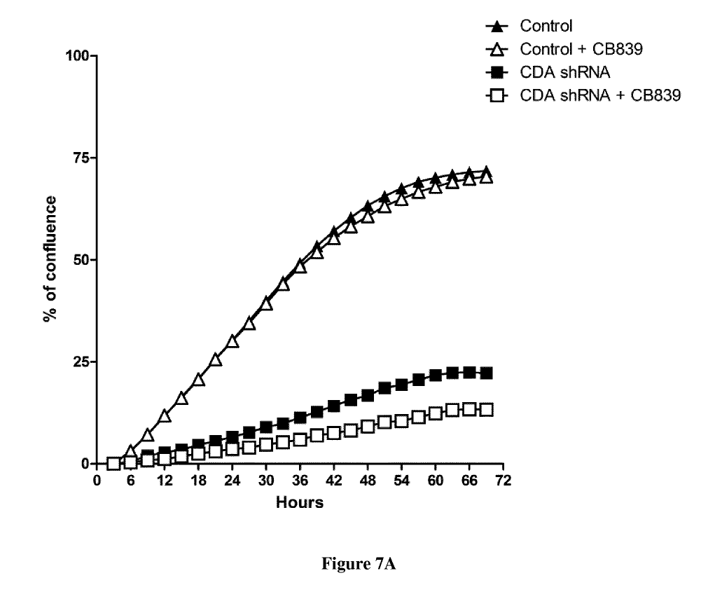

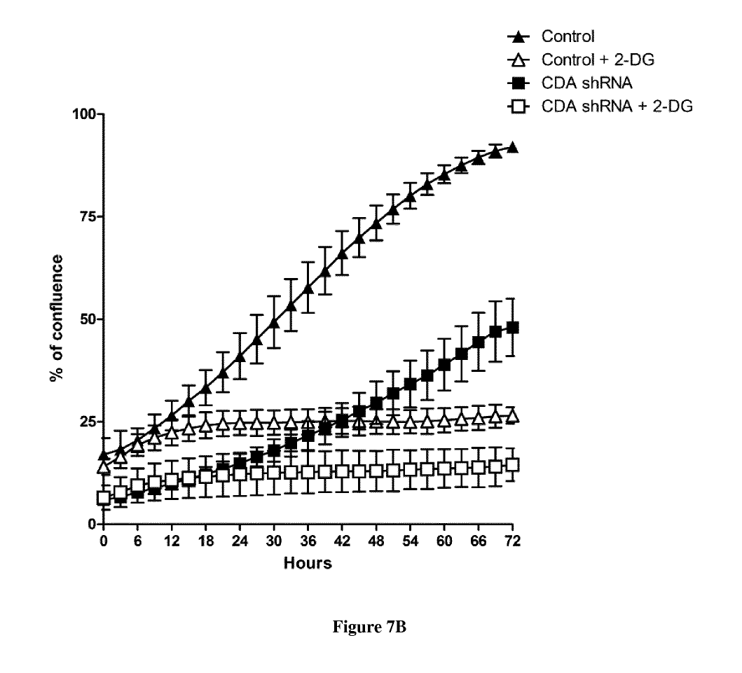

While complementary experiments are still needed, our results strongly suggest an exquisite addiction of CDA-null PDA cells to glutamine. This finding opens several other combinatory therapeutic options as glutaminase (CB-839, FIG. 7) and α-KG dehydrogenase (CPI-613, a lipoate analog, inhibits mitochondrial enzymes pyruvate dehydrogenase (PDH) and α-KG dehydrogenase in NCI-H460 cell line, disrupts tumor cell mitochondrial metabolism) inhibitors are being evaluated clinically for PDA subjects⁵³. The redox potential of CDA-depleted cells could be severely altered, as we measured a decrease in 02 consumption, an increase of mitochondrial ROS with concomitant decrease of GSH/GSSG ratio (FIG. 4); remarkably, we identified adenosylhomocysteinase (AHCY) as upregulated in CDA-null cells probably to compensate the deleterious effects of CDA depletion by upregulating GSH levels, offering new targeting possibilities using drugs against this enzyme (3-deazaneplanocine A, DZNe) in combination with small inhibitors against CDA.

### REFERENCES

Throughout this application, various references describe the state of the art to which this invention pertains. The disclosures of these references are hereby incorporated by reference into the present disclosure.
1. Vincent, A., Herman, J., Schulick, R., Hruban, R. H. & Goggins, M. Pancreatic cancer. Lancet Lond. Engl. 378, 607-620 (2011).
2. Rahib, L. et al. Projecting cancer incidence and deaths to 2030: the unexpected burden of thyroid, liver, and pancreas cancers in the United States. Cancer Res. 74, 2913-2921 (2014).
3. Conroy, T. et al. FOLFIRINOX versus gemcitabine for metastatic pancreatic cancer. N. Engl. J. Med. 364, 1817-1825 (2011).
4. Burris, H. A., 3rd et al. Improvements in survival and clinical benefit with gemcitabine as first-line therapy for patients with advanced pancreas cancer: a randomized trial. J. Clin. Oncol. Off. J. Am. Soc. Clin. Oncol. 15, 2403-2413 (1997).
5. Von Hoff, D. D. et al. Increased survival in pancreatic cancer with nab-paclitaxel plus gemcitabine. N. Engl. J. Med. 369, 1691-1703 (2013).
6. Moore, M. J. et al. Erlotinib plus gemcitabine compared with gemcitabine alone in patients with advanced pancreatic cancer: a phase III trial of the National Cancer Institute of Canada Clinical Trials Group. J. Clin. Oncol. Off. J. Am. Soc. Clin. Oncol. 25, 1960-1966 (2007).
7. Vassaux, G. et al. The promise of gene therapy for pancreatic cancer. Hum. Gene Ther. (2015). doi:10.1089/hum.2015.141
8. Buscail, L. et al. First-in-man phase I clinical trial of gene therapy for advanced pancreatic cancer: Safety, biodistribution and preliminary clinical findings. Mol. Ther. J. Am. Soc. Gene Ther. (2015). doi:10.1038/mt.2015.1
9. Nygaard, P. On the role of cytidine deaminase in cellular metabolism. Adv. Exp. Med. Biol. 195 Pt B, 415-420 (1986).
10. Samulitis, B. K. et al. Gemcitabine resistant pancreatic cancer cell lines acquire an invasive phenotype with collateral hypersensitivity to histone deacetylase inhibitors. Cancer Biol. Ther. 16, 43-51 (2015).
11. Avan, A. et al. Enhancement of the antiproliferative activity of gemcitabine by modulation of c-Met pathway in pancreatic cancer. Curr. Pharm. Des. 19, 940-950 (2013).
12. Funamizu, N. et al. Tetrahydrouridine inhibits cell proliferation through cell cycle regulation regardless of cytidine deaminase expression levels. PloS One 7, e37424 (2012).
13. Funamizu, N. et al. Is the resistance of gemcitabine for pancreatic cancer settled only by overexpression of deoxycytidine kinase? Oncol. Rep. 23, 471-475 (2010).
14. Weizman, N. et al. Macrophages mediate gemcitabine resistance of pancreatic adenocarcinoma by upregulating cytidine deaminase. Oncogene (2013). doi:10.1038/onc.2013.357
15. Frese, K. K. et al. nab-Paclitaxel potentiates gemcitabine activity by reducing cytidine deaminase levels in a mouse model of pancreatic cancer. Cancer Discov. 2, 260-269 (2012).
16. Scotland, S. et al. Mitochondrial energetic and AKT status mediate metabolic effects and apoptosis of metformin in human leukemic cells. Leukemia 27, 2129-2138 (2013).
17. Sesen, J. et al. Metformin inhibits growth of human glioblastoma cells and enhances therapeutic response. PloS One 10, e0123721 (2015).
18. Basturk, O. et al. A Revised Classification System and Recommendations From the Baltimore Consensus Meeting for Neoplastic Precursor Lesions in the Pancreas. Am. J. Surg. Pathol. 39, 1730-1741 (2015).
19. Ligat, L. et al. Pancreatic preneoplastic lesions plasma signatures and biomarkers based on proteome profiling of mouse models. Br. J. Cancer (2015). doi: 10.1038/bjc.2015.370
20. Bournet, B. et al. Role of endoscopic ultrasound in the molecular diagnosis of pancreatic cancer. World J. Gastroenterol. WJG 20, 10758-10768 (2014).
21. Hingorani, S. R. et al. Preinvasive and invasive ductal pancreatic cancer and its early detection in the mouse. Cancer Cell 4, 437-450 (2003).
22. du Rieu, M. C. et al. MicroRNA-21 is induced early in pancreatic ductal adenocarcinoma precursor lesions. Clin. Chem. 56, 603-612 (2010).
23. Hanoun, N. et al. The silencing of microRNA 148a production by DNA hypermethylation is an early event in pancreatic carcinogenesis. Clin. Chem. 56, 1107-1118 (2010).
24. Scarpa, A. et al. Pancreatic adenocarcinomas frequently show p53 gene mutations. Am. J. Pathol. 142, 1534-1543 (1993).
25. Bailey, J. M. et al. p53 mutations cooperate with oncogenic Kras to promote adenocarcinoma from pancreatic ductal cells. Oncogene (2015). doi: 10.1038/onc.2015.441
26. Hingorani, S. R. et al. Trp53R172H and KrasG12D cooperate to promote chromosomal instability and widely metastatic pancreatic ductal adenocarcinoma in mice. Cancer Cell 7, 469-483 (2005).
27. Mahfouz, R. Z. et al. Increased CDA expression/activity in males contributes to decreased cytidine analogue half-life and likely contributes to worse outcomes with 5-azacytidine or decitabine therapy. Clin. Cancer Res. Off. J. Am. Assoc. Cancer Res. 19, 938-948 (2013).
28. Ye, F.-G. et al. Cytidine Deaminase Axis Modulated by miR-484 Differentially Regulates Cell Proliferation and Chemoresistance in Breast Cancer. Cancer Res. 75, 1504-1515 (2015).
29. Kong, X. et al. Detection of differentially expressed microRNAs in serum of pancreatic ductal adenocarcinoma patients: miR-196a could be a potential marker for poor prognosis. Dig Sci 56, 602-9 (2011).
30. Gayral, M. et al. MicroRNAs as emerging biomarkers and therapeutic targets for pancreatic cancer. World J. Gastroenterol. WJG 20, 11199-11209 (2014).
31. Gayral, M., Torrisani, J. & Cordelier. in microRNAs in Toxicology and Medicine 167-172 (2013).
32. Li, A. et al. MicroRNA array analysis finds elevated serum miR-1290 accurately distinguishes patients with low-stage pancreatic cancer from healthy and disease controls. Clin. Cancer Res. Off. J. Am. Assoc. Cancer Res. 19, 3600-3610 (2013).
33. Mei, Q. et al. Methylation-induced loss of miR-484 in microsatellite-unstable colorectal cancer promotes both viability and IL-8 production via CD137L. J. Pathol. 236, 165-174 (2015).
34. Delpu, Y. et al. The Rescue of miR-148a Expression in Pancreatic Cancer: An Inappropriate Therapeutic Tool. PLoS ONE 8, e55513 (2013).
35. Torrisani, J. et al. let-7 MicroRNA transfer in pancreatic cancer-derived cells inhibits in vitro cell proliferation but fails to alter tumor progression. Hum. Gene Ther. 20, 831-844 (2009).
36. Chiou, S.-H. et al. Pancreatic cancer modeling using retrograde viral vector delivery and in vivo CRISPR/Cas9-mediated somatic genome editing. Genes Dev. 29, 1576-1585 (2015).
37. Mazur, P. K. et al. Combined inhibition of BET family proteins and histone deacetylases as a potential epigenetics-based therapy for pancreatic ductal adenocarcinoma. Nat. Med. 21, 1163-1171 (2015).
38. Guerra, C. et al. Pancreatitis-induced inflammation contributes to pancreatic cancer by inhibiting oncogene-induced senescence. Cancer Cell 19, 728-739 (2011).
39. Duconseil, P. et al. Transcriptomic analysis predicts survival and sensitivity to anticancer drugs of patients with a pancreatic adenocarcinoma. Am. J. Pathol. 185, 1022-1032 (2015).
40. Aguilera, A. & Garcia-Muse, T. Causes of genome instability. Annu. Rev. Genet. 47, 1-32 (2013).
41. Tourribre, H. & Pasero, P. Maintenance of fork integrity at damaged DNA and natural pause sites. DNA Repair 6, 900-913 (2007).
42. Couch, F. B. et al. ATR phosphorylates SMARCAL1 to prevent replication fork collapse. Genes Dev. 27, 1610-1623 (2013).
43. Allen, C., Ashley, A. K., Hromas, R. & Nickoloff, J. A. More forks on the road to replication stress recovery. J. Mol. Cell Biol. 3, 4-12 (2011).
44. Aze, A., Zhou, J. C., Costa, A. & Costanzo, V. DNA replication and homologous recombination factors: acting together to maintain genome stability. Chromosoma 122, 401-413 (2013).
45. Murga, M. et al. Exploiting oncogene-induced replicative stress for the selective killing of Myc-driven tumors. Nat. Struct. Mol. Biol. 18, 1331-1335 (2011).
46. Gemble, S. et al. Pyrimidine Pool Disequilibrium Induced by a Cytidine Deaminase Deficiency Inhibits PARP-1 Activity, Leading to the Under Replication of DNA. PLoS Genet. 11, e1005384 (2015).
47. Harrigan, J. A. et al. Replication stress induces 53BP1-containing OPT domains in G1 cells. J. Cell Biol. 193, 97-108 (2011).
48. Lukas, C. et al. 53BP1 nuclear bodies form around DNA lesions generated by mitotic transmission of chromosomes under replication stress. Nat. Cell Biol. 13, 243-253 (2011).
49. Naim, V. & Rosselli, F. The FANC pathway and BLM collaborate during mitosis to prevent micro-nucleation and chromosome abnormalities. Nat. Cell Biol. 11, 761-768 (2009).
50. Bétous, R. et al. DNA polymerase K-dependent DNA synthesis at stalled replication forks is important for CHK1 activation. EMBO J. 32, 2172-2185 (2013).
51. Bergoglio, V. et al. DNA synthesis by Pol η promotes fragile site stability by preventing under-replicated DNA in mitosis. J. Cell Biol. 201, 395-408 (2013).
52. Femandez-Vidal, A. et al. A role for DNA polymerase θ in the timing of DNA replication. Nat. Commun. 5, 4285 (2014).
53. Perera, R. M. & Bardeesy, N. Pancreatic Cancer Metabolism: Breaking It Down to Build It Back Up. Cancer Discov. (2015). doi:10.1158/2159-8290.CD-15-0671
54. Ying, H. et al. Oncogenic Kras maintains pancreatic tumors through regulation of anabolic glucose metabolism. Cell 149, 656-670 (2012).
55. DeNicola, G. M. et al. Oncogene-induced Nrf2 transcription promotes ROS detoxification and tumorigenesis. Nature 475, 106-109 (2011).
56. Aarts, M. et al. Forced mitotic entry of S-phase cells as a therapeutic strategy induced by inhibition of WEE1. Cancer Discov. 2, 524-539 (2012).
57. Meier, F. M. P., Frerix, M., Hermann, W. & Müller-Ladner, U. Current immunotherapy in rheumatoid arthritis. Immunotherapy 5, 955-974 (2013).

1. A method for treating pancreatic cancer in a subject in need thereof comprising administering to the subject a therapeutically effective amount of a cytidine deaminase inhibitor in combination with an anti-pancreatic cancer treatment selected from the group consisting of CHK1 inhibitor, WEE1 inhibitor, ART inhibitor, DHODH inhibitor and gene therapy.

2. The method of claim 1 wherein the cytidine deaminase inhibitor is selected from the group consisting of fluorinated tetrahydrouridines and derivatives thereof, 2′-fluorinated tetrahydrouridine derivatives and difluorotetrahydrouridine derivatives.

3. The method of claim 1 wherein the cytidine deaminase inhibitor is selected from the group consisting of ASTX727 (E7727); 5-methyl-2′,3′-dideoxy-3′-azidocytidine (5mAZC); 5-methyl-2′,3′-dideoxycytidine; 5-ethyl-2′,3′dideoxy-3′-azidocytidine; 5-propyl-2′,3′-dideoxycytidine; 5-propyl-2′,3′-dideoxy-3′-azidocytidine; 5-propene-2′,3′-dideoxy-3′-azidocytidine; 5-propyne-2′,3′-dideoxy-3′-azidocytidine; 5-propyne-2′,3′-dideoxy-3′-azidocytidine; and Zebularine (1-(β-D-Ribofuranosyl)-2(1H)-pyrimidinone), or analogues or pharmaceutically effective salts thereof.

4. The method of claim 1 wherein the cytidine deaminase inhibitor is an inhibitor of cytidine deaminase expression.

5. The method of claim 4 wherein the inhibitor of cytidine deaminase expression is an antisense oligonucleotide.

6. The method of claim 4 wherein the inhibitor of cytidine deaminase expression is delivered in association with a vector selected from the group consisting of plasmid, phagemid, non-cytopathic virus and oncolytic virus.

7. A method for treating pancreatic cancer in a subject in need thereof comprising administering to the subject a therapeutically effective amount of a cytidine deaminase inhibitor in combination with DCK::UMK fusion gene therapy and gemcitabine.

8. A method of screening a candidate compound for use as a drug for treating pancreatic cancer in a subject in need thereof, wherein positively selected candidate compounds inhibit cytidine deaminase (CDA), wherein the method comprises the steps of:
providing a cytidine deaminase (CDA) or a cell, tissue sample or organism expressing the CDA,
providing a candidate compound,
measuring CDA activity in the presence of the candidate compound, and and selecting positively candidate compounds that inhibit the CDA activity.

9. The method of claim 8, wherein the candidate compound is a small organic molecule, a polypeptide, an aptamer, an antibody or an intra-antibody.
